(19) United States
(12) Patent Application Publication
(10) Pub. No.: US 2014/0345641 A1
(43) Pub. Date: Nov. 27, 2014

(54) PRODUCTS FOR TEMPORARILY SHAPING KERATIN FIBERS AND METHODS FOR TEMPORARILY SHAPING HAIR

(71) Applicant: Henkel AG & Co. KGaA, Dusseldorf (DE)

(72) Inventors: Thorsten Knappe, Schenefeld (DE); Helga van Flodrop, Duesseldorf (DE)

(21) Appl. No.: 14/364,599

(22) PCT Filed: Nov. 9, 2012
(86) PCT No.: PCT/EP2012/072221

§ 371 Date: Jun. 11, 2014

(30) Foreign Application Priority Data
Dec. 16, 2011 (DE) 102011088844.6

(51) Int. Cl. A61K 8/85; A45D 7/06; A61Q 5/06; A61Q 5/12; A61K 8/84; A61K 8/41

(52) U.S. Cl. CPC A61K 8/85; A61K 8/84; A61K 8/416; A61Q 5/06; A61Q 5/12; A45D 7/06; A61K 2800/54
USPC 132/206; 424/70.11

(57) ABSTRACT

A cosmetic agent containing in a cosmetically acceptable carrier
a) at least one copolymer A with at least one structural unit (A1), at least one structural unit (A2), at least one structural unit (A3) and at least one structural unit (A4),

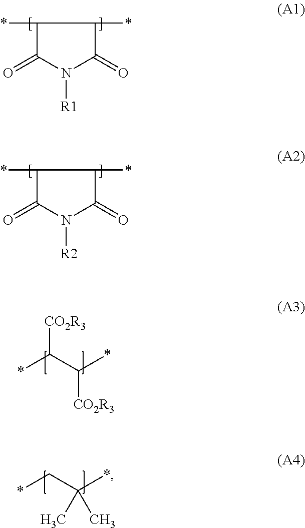

wherein
R1 denotes an optionally heterofunctionalized alkyl residue;
R2 denotes an optionally heterofunctionalized alkyl residue differing from R1;
R3 denotes an optionally heterofunctionalized alkyl residue mutually independently differing from R1 and R2
b) at least one copolymer B differing from copolymer A with at least one structural unit (B1)

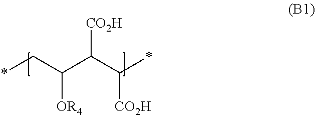

wherein R4 denotes an optionally heterofunctionalized alkyl residue; and
c) at least one quaternary ammonium compound.

### CROSS REFERENCE TO RELATED APPLICATIONS

The present disclosure is a U.S. National Stage entry under 35 U.S.C. §371 based on International Application No. PCT/EP2012/072221, filed Nov. 9, 2012 which was published under PCT Article 21(2) and which claims priority to German Patent Application No. 10 2011 088 844.6 filed on Dec. 16, 2011, which is incorporated herein by reference in its entirety.

### TECHNICAL FIELD

The technical field relates to cosmetic preparations for temporarily deforming hair, which cosmetic preparations are particularly suitable for temporarily deforming hair with exposure to heat, and to the use of such preparations for deforming hair and to methods for temporarily deforming hair.

### BACKGROUND

Keratinic fibers, in particular human hair, are these days subjected to a multiplicity of treatments. Treatments which bring about permanent or temporary shaping of the hair play an important role. Current fashion trends often mean that, with many hair types, hairstyles which are considered stylish can only be achieved or maintained for an extended period by using specific setting active substances. Examples of temporary shaping operations are curling, straightening, back-combing or indeed setting. Temporary shaping, which is intended to provide a good hold without impairing the healthy appearance of the hair, such as for example the gloss thereof, may be achieved for example by styling agents such as hairsprays, hair waxes, hair gels, hair setting preparations, setting lotions, styling sprays etc. Temporary shaping is also known as hair styling or styling, while shaping agents are also known as styling agents.

In the field of temporary shaping of hair, hair deformation with exposure to heat, for example using a hairdryer, curling tongs or hair straighteners likewise play an important part. To improve styling results, a styling agent, for example a hair setting preparation or a styling spray or in the case of relatively high temperatures a “heat styling spray”, is often applied to the hair before heat is applied. Hairdressers in particular are making increasing use of hair straighteners (or “straightening irons”). Hair straighteners comprise two metal or ceramic plates arranged parallel to one another, through which the hair is drawn once the plates have been heated, the hair straighteners being passed over a strand of hair. Conventional commercial hair straighteners may be heated up to temperatures in the range from 150-250° C. The purpose of using hair straighteners is to straighten wavy to curly hair physically/using heat. If hair is to be straightened using hair straighteners, a heat styling spray, also known as straightening spray, is usually applied to the hair beforehand as a hair straightener styling agent. The spray helps the straighteners to slide and assists straightening of the hair.

Hair deformation through exposure to heat is also used to achieve the opposite effect, namely waving or curling straight hair. In this case, instead of hair straighteners, curling tongs or indeed a hairdryer with diffuser are used. To do this, the straight hair is wound around the heated curling tongs or the diffuser, wherein temperatures of up to 250° C. are once again reached. In this case too, heat styling sprays which are applied to the hair before the actual heat treatment are usually used to improve the styling result.

In previously described hair deformation methods, the exposure to heat unfortunately not only achieves the desired cosmetic action, but also has a generally negative effect on the stability, tactile properties and visual appearance of the keratin-containing fibers, in particular the human hair. Hair damaged in this manner appears dull and brittle. In extreme cases, hair breakage may even occur. Against this background, there is a need to provide heat styling sprays which on the one hand counteract the described disadvantages while on the other hand having not only hair-conditioning but also hair-fixing properties, such that the duration of and/or the level of exposure to heat during thermal hair deformation may be reduced. The prior art discloses numerous cosmetic compositions for temporary hair deformation which are also for use with exposure to heat but do not fulfill any of the above-stated criteria. An additional disadvantage of the cosmetic compositions known from the prior art for temporarily deforming hair with exposure to heat is that an intense unpleasant odor often develops when heat is applied.

Accordingly, it is desirable to provide a safe cosmetic composition for hair treatment with exposure to heat, i.e. a toxicologically safe composition which remains toxicologically safe even at temperatures of up to 200° C. It also is desirable to provide a cosmetic composition for hair treatment with exposure to heat at temperatures in the range of up to 200° C. which exhibits a markedly improved conditioning action, improved shaping and reduced odor development compared with conventional compositions when applied at these temperatures. Furthermore, other desirable features and characteristics will become apparent from the subsequent detailed description of the invention and the appended claims, taken in conjunction with the accompanying drawings and this background of the invention.

### SUMMARY

Cosmetic agents and methods for temporarily deforming keratinic fibers are provided herein. In an exemplary embodiment, a cosmetic agent contains in a cosmetically acceptable carrier
a) at least one copolymer A with at least one structural unit (A1), at least one structural unit (A2), at least one structural unit (A3) and at least one structural unit (A4).

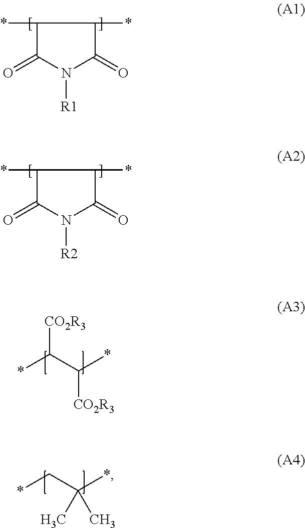

wherein
   R1 denotes an optionally heterofunctionalized alkyl residue;
   R2 denotes an optionally heterofunctionalized alkyl residue differing from R1;
   R3 denotes an optionally heterofunctionalized alkyl residue mutually independently differing from R1 and R2
b) at least one copolymer B differing from copolymer A with at least one structural unit (B1)

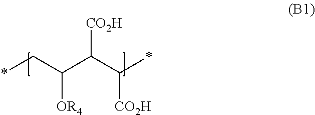

wherein R4 denotes an optionally heterofunctionalized alkyl residue; and
c) at least one quaternary ammonium compound.

In another exemplary embodiment, a method for temporarily deforming keratinic fibers includes:
applying onto the keratinic fibers a cosmetic agent containing in a cosmetically acceptable carrier
   a) at least one copolymer A with at least one structural unit (A1), at least one structural unit (A2), at least one structural unit (A3) and at least one structural unit (A4),

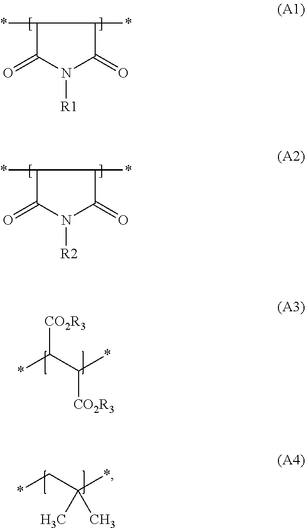

wherein
   R1 denotes an optionally heterofunctionalized alkyl residue;
   R2 denotes an optionally heterofunctionalized alkyl residue differing from R1;
   R3 denotes an optionally heterofunctionalized alkyl residue mutually independently differing from R1 and R2
b) at least one copolymer B differing from copolymer A with at least one structural unit (B1)

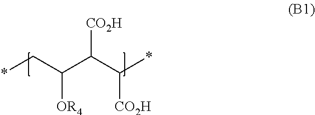

wherein R4 denotes an optionally heterofunctionalized alkyl residue; and
c) at least one quaternary ammonium compound.

### DETAILED DESCRIPTION

Various embodiments of cosmetic compositions contemplated herein contain a specific active ingredient combination of polymers and quaternary ammonium compounds. An exemplary embodiment provides a cosmetic agent, containing in a cosmetically acceptable carrier

a) at least one copolymer A with at least one structural unit (A1), at least one structural unit (A2), at least one structural unit (A3) and at least one structural unit (A4),

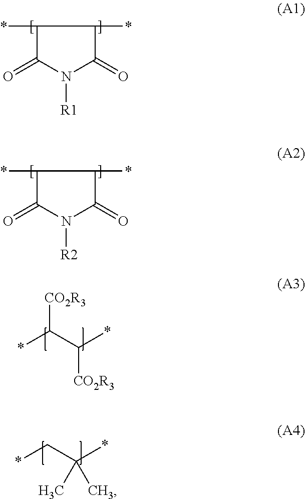

wherein
R1 denotes an optionally heterofunctionalized alkyl residue;
R2 denotes an optionally heterofunctionalized alkyl residue differing from R1;
R3 denotes an optionally heterofunctionalized alkyl residue mutually independently differing from R1 and R2
   b) at least one copolymer B differing from copolymer A with at least one structural unit (B1)

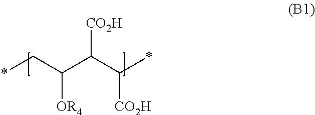

wherein R4 denotes an optionally heterofunctionalized alkyl residue; and
c) at least one quaternary ammonium compound.

The agents contemplated herein contain the active substances in a cosmetic carrier. This cosmetic carrier is aqueous, alcoholic or aqueous-alcoholic. For the purposes herein, aqueous-alcoholic carriers should be taken to be hydrous compositions containing about 3 to about 70 wt. % of a C₁-C₄ alcohol, in particular ethanol or isopropanol, relative to the total weight of the mixture for use. For the purposes herein, an aqueous carrier contains at least about 30 wt. %, in particular at least about 50 wt. % water, relative to the total weight of the mixture for use. Preferred cosmetic agents contain, relative to the total weight thereof, about 40 to about 99 wt. %, preferably about 50 to about 98 wt. %, more preferably about 60 to about 95 wt. % and in particular about 70 to about 90 wt. % water. Particularly preferred cosmetic agents comprise about 60 to about 99 wt. %, preferably about 75 to about 98 wt. % and in particular about 85 to about 95 wt. % water and about 0.1 to about 20 wt. %, preferably about 0.5 to about 10 wt. % and in particular about 1.0 to about 7.0 wt. % C₁-C₄-alcohol, preferably ethanol.

The agents contemplated herein contain as first essential component a copolymer A, comprising the structural units (A1), (A2), (A3) and (A4). With regard to the technical effect of the agents contemplated herein, it has proven advantageous for copolymer A to consist in a proportion of at least about 70 wt. %, preferably at least about 80 wt. %, preferably at least about 90 wt. % and in particular at least about 95 wt. % of structural units (A1), (A2), (A3) and (A4). Further preferred copolymers A consist completely of structural units (A1), (A2), (A3) and (A4).

In one preferred embodiment, the residue R1 in structural unit (A1) denotes an ether residue, preferably a polyalkoxylated residue. Structural units (A1) which are more preferred are those in which R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100.

The residue R2 in the structural unit (A2) preferably denotes an amino group-containing residue, preferably a residue with a tertiary amine. Structural units (A2) which are more preferred are those in which R2 in formula (A2) denotes a residue —(CH₂)₃—N(CH₃)₂.

In structural unit (A3) preferably at least one alkyl residue, preferably a C₁ to C₄ alkyl residue is used as residue R3. Structural units (A3) which are more preferred are those in which R3 denotes —CH₂CH₃ or —CH₂CH₂CH₃, preferably —CH₂CH₃ and a residue R3 in formula (A3) denotes H.

Some preferred copolymers A are listed below. These copolymers A consist in a proportion of at least about 70 wt. %, preferably of at least about 80 wt. %, preferably of at least about 90 wt. % and in particular of at least about 95 wt. %, particularly preferably completely of structural units (A1), (A2), (A3) and (A4):

A-I) copolymer A with at least one structural unit (A1), at least one structural unit (A2), at least one structural unit (A3) and at least one structural unit (A4),

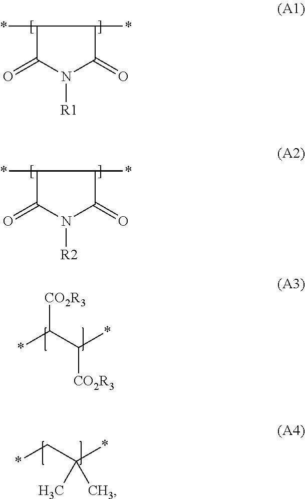

wherein

R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100;

R2 in formula (A2) denotes a residue —(CH₂)₃—N(CH₃)₂;

R3 denotes an optionally heterofunctionalized alkyl residue mutually independently differing from R1 and R2.

A-II) copolymer A with at least one structural unit (A1), at least one structural unit (A2), at least one structural unit (A3) and at least one structural unit (A4),

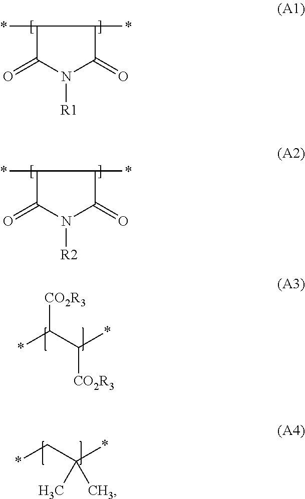

wherein

R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100;

R2 denotes an optionally heterofunctionalized alkyl residue differing from R1;

a residue R3 in the formula (A3) denotes —CH₂CH₃ or —CH₂CH₂CH₃, preferably —CH₂CH₃ and a residue R3 in formula (A3) denotes H.

A-III) copolymer A with at least one structural unit (A1), at least one structural unit (A2), at least one structural unit (A3) and at least one structural unit (A4),

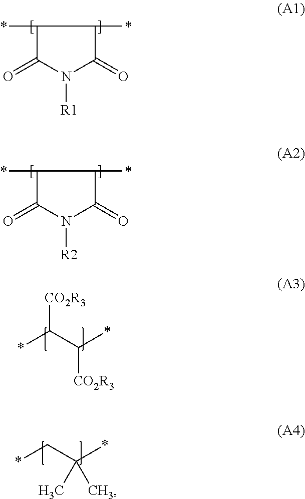

wherein

R1 denotes an optionally heterofunctionalized alkyl residue;

R2 in formula (A2) denotes a residue —(CH₂)₃—N(CH₃)₂;

a residue R3 in the formula (A3) denotes —CH₂CH₃ or —CH₂CH₂CH₃, preferably —CH₂CH₃ and a residue R3 in formula (A3) denotes H.

Particularly preferred cosmetic agents are characterized in that

the residue R1 in formula (A1) denotes a residue —CH(CH₃)CH₂—(OCH(CH₃)CH₂)ₓ(O[CH₂]₂)_{y}OCH₃, in which x and y mutually independently have a value of between 1 and 100;

the residue R2 in formula (A2) denotes a residue —(CH₂)₃—N(CH₃)₂;

one residue R3 denotes —CH₂CH₃ and one residue R3 in formula (A3) denotes H.

The proportion by weight of copolymer A in the total weight of cosmetic agents contemplated herein preferably amounts to about 0.05 to about 10 wt. %, preferably about 0.1 to about 7.0 wt. % and in particular about 0.2 to about 5.0 wt. %.

As their second essential component, the agents contemplated herein contain a copolymer B, comprising the structural unit (B1). With regard to the technical effect of the agents, it has proven advantageous for copolymer B to consist in a proportion of at least about 70 wt. %, preferably at least about 80 wt. %, preferably at least about 90 wt. % and in particular at least about 95 wt. % of structural unit (B1). Further preferred copolymers B consist completely of structural unit (B1).

In a preferred embodiment, the residue R4 in structural unit (B1) denotes an alkyl residue, preferably a C₁ to C₄ alkyl residue. The structural unit (B1) in which residue R4 denotes —CH₃ is more preferred.

The proportion by weight of copolymer B in the total weight of a cosmetic agent preferably amounts to about 0.05 to about 10 wt. %, preferably about 0.1 to about 7.0 wt. % and in particular about 0.2 to about 5.0 wt. %.

Copolymers A and B may be incorporated into the agent contemplated herein in pure form. For processing and the cosmetic properties of the cosmetic agents, however, it has proven advantageous to use copolymers A and B in pre-formulated form, i.e. in combination with further active or auxiliary substances. In particular, mixtures of copolymers A and B are preferably used. The weight ratio of copolymer A to copolymer B in the polymer mixtures preferably used amounts of from about 20:1 to about 1:20, preferably about 10:1 to about 1:10, in particular about 8:1 to about 1:8 and particularly preferably about 5:1 to about 1:5. Consequently, preferred cosmetic agents contemplated herein are therefore also those in which the weight ratio of copolymer A to copolymer B are from about 20:1 to about 1:20, preferably about 10:1 to about 1:10, in particular about 8:1 to about 1:8 and particularly preferably about 5:1 to about 1:5. The use of copolymers A and B in a weight ratio of about 2:1 to about 1:4, preferably of about 1:1 to about 1:3 is particularly preferred, with copolymer B preferably being used in excess.

The previously described copolymers A and B or mixtures thereof are preferably combined with additional auxiliary substances. The use of alcohols is more preferred. A preferred class of alcohols is diols, in particular 1,2-diols. 1,2-Octanediol is particularly preferentially used. In particular, 1,2-octanediol not only simplifies further processing of copolymers A and B, or mixtures thereof, but also enhances the advantageous technical effect thereof, in particular the conditioning effect thereof.

The above-stated diols may be incorporated into the cosmetic agents contemplated herein together with copolymers A and B, or mixtures thereof, or separately from copolymers A and B. Against this background, preferred cosmetic agents are those which additionally contain at least one 1,2-diol, preferably 1,2-octanediol.

In an exemplary embodiment, a polymer mixture based on copolymers A and B is distributed by ISP under the name Styleze® XT3 (INCI: Water (and) Polyimide-1 (and) PVM/MA Copolymer (and) Caprylyl Glycol (proposed)).

To summarize, particularly preferred cosmetic agents contain

a) at least one copolymer A with at least one structural unit (A1), at least one structural unit (A2), at least one structural unit (A3) and at least one structural unit (A4),

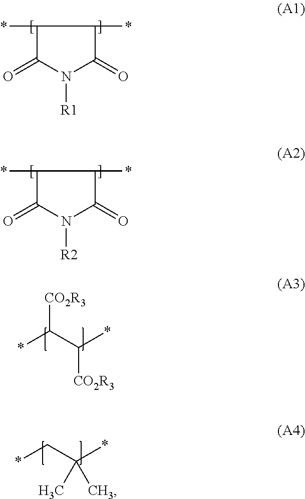

wherein

R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100;

R2 in formula (A2) denotes a residue —(CH₂)₃—N(CH₃)₂;

one residue R3 denotes —CH₂CH₃ and one residue R3 in formula (A3) denotes H;

b) at least one copolymer B differing from copolymer A with at least one structural unit (B1)

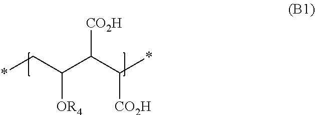

wherein R4 denotes —CH₃;
c) at least one quaternary ammonium compound.

The cosmetic agents contemplated herein contain as further essential component at least one quaternary ammonium compound. Monomeric or polymeric active substances may be used as the quaternary ammonium compound.

From the plurality of possible monomeric quaternary ammonium compounds, the compounds from the groups:

trimethylalkylammonium halides;

ester quats

quaternary imidazolines

have proven particularly effective.

The group of trimethylalkylammonium halides in particular includes the compounds of formula (Tkat1-1).

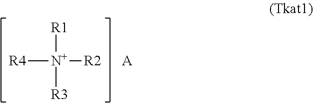

In the formula (Tkat1), R1, R2, R3 and R4 in each case mutually independently denote hydrogen, a methyl group, a phenyl group, a benzyl group, a saturated, branched or unbranched alkyl residue with a chain length of 8 to 30 carbon atoms, which may optionally be substituted with one or more hydroxyl groups. A denotes a physiologically acceptable anion, for example halides such as chloride or bromide and methosulfates.
Examples of compounds of formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetyl-methylammonium chloride, stearyltrimethylammonium chloride, distearyl-dimethylammonium chloride, lauryldimethylbenzylammonium chloride, behenyltrimethylammonium chloride, behenyltrimethylammonium bromide, behenyltrimethylammonium methosulfate. Preferred cosmetic agents contain a monomeric quaternary ammonium compound from the group of trimethylalkylammonium halides.

Further quaternary ammonium compounds which are more preferred are the cationic betaine esters of formula (Tkat1-2.1).

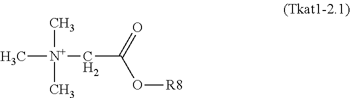

More preferred ester quats are those with the trade names Armocare VGH-70, and Dehyquart® F-75, Dehyquart® L80, Stepantex® VS 90 and Akypoquat® 131.

A further group are quaternary imidazoline compounds. The formula (Tkat2) illustrated below shows the structure of these compounds.

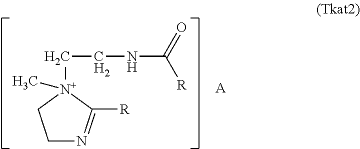

The residues R mutually independently in each case denote a saturated or unsaturated, linear or branched hydrocarbon residue with a chain length of 8 to 30 carbon atoms. The preferred compounds of the formula (Tkat2) in each case contain the identical hydrocarbon residue for R. The chain length of the residues R preferably amounts to 12 to 21 carbon atoms. A denotes an anion as previously described. Examples which are particularly suitable are obtainable for example under NCI names Quaternium-27, Quaternium-72, Quaternium-83, Quaternium-87 and Quaternium-91. Quaternium-91 is most preferred.

With regard to cosmetic action, advantageous cosmetic agents have proven to be those in which the proportion by weight of the monomeric quaternary ammonium compound in the total weight of the agent amounts to from about 0.05 to about 3.0 wt. %, preferably about 0.1 to about 2.0 wt. % and in particular about 0.2 to about 1.0 wt. %.

In preferred cosmetic agents, the ratio of the joint proportion by weight of polymers A and B to the proportion by weight of the monomeric quaternary ammonium compound c) amounts to between about 8:1 and about 1:8, preferably between about 6:1 and about 1:6 and in particular between about 4:1 and about 1:4.

In an exemplary embodiment, the group of polymeric quaternary ammonium compounds include the cationic polymers. These polymers comprise a quaternary, i.e. permanently cationic, nitrogen atom with four substituents. The polymeric quaternary ammonium compounds in other words comprise polymers with a “permanently” cationic ammonium compound. Polymers which are designated “permanently cationic” herein are those which, irrespective of the pH value of the agent, comprise a cationic group. Preferred cationic groups are quaternary ammonium groups which are attached via a C₁₋₄ hydrocarbon group to a polymer main chain consisting of acrylic acid, methacrylic acid or the derivatives thereof.

Preferred polymers are those which have sufficient solubility in water or alcohol in order to pass completely into solution in the agent contemplated herein.

The cationic polymers may be homo- or copolymers, wherein the quaternary nitrogen groups are present either in the polymer chain or preferably as substituents on one or more of the monomers. Suitable cationic monomers are unsaturated, free-radically polymerizable compounds which bear at least one cationic, quaternary ammonium group, in particular ammonium-substituted vinyl monomers such as for example trialkylmethacryloxyalkylammonium, trialkylacryloxyalkylammonium, dialkyldiallyl-ammonium and quaternary vinylammonium monomers with cyclic, cationic nitrogen-containing groups such as pyridinium, imidazolium or quaternary pyrrolidones, for example alkylvinylimidazolium, alkylvinylpyridinium or alkylvinylpyrrolidone salts. The alkyl groups of these monomers are preferably lower alkyl groups such as for example C₁ to C₇ alkyl groups, more preferably C₁ to C₃ alkyl groups.

Monomers containing ammonium groups may be copolymerized with non-cationic monomers. Suitable comonomers are for example acrylamide, methacrylamide; alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, wherein the alkyl groups of these monomers are preferably C₁ to C₇ alkyl groups, more preferably C₁ to C₃ alkyl groups.

Suitable polymers with quaternary ammonium groups are for example the polymers described in the CTFA Cosmetic Ingredient Dictionary under the names Polyquaternium, such as methylvinylimidazolium chloride/vinylpyrrolidone copolymer (Polyquaternium-16) or quaternized vinylpyrrolidone/dimethylaminoethyl methacrylate copolymer (Polyquaternium-11).

Further examples of polymeric quaternary ammonium compounds are

copolymers of vinylpyrrolidone with quaternized derivatives of dialkylaminoalkylacrylate and -methacrylate, for example vinyl-pyrrolidone/dimethylaminoethylmethacrylate methosulfate copolymers, as distributed by Gaf Co., USA under the trade names Gafquat® 755 N and Gafquat® 734;

polyvinylpyrrolidone/imidazolimine methochloride copolymers, as distributed by BASF, Germany under the trade name Luviquat® MQ 550;

terpolymers of dimethyldiallylammonium chloride, sodium acrylate and acrylamide, distributed by Calgon/USA under the trade name Merquat® Plus 3300;

vinylpyrrolidone/methacrylamidopropyltrimethylammonium chloride copolymer, distributed by ISP under the trade name Gafquat® HS 100;

polymeric dimethyldiallylammonium salts and the copolymers thereof with esters and amides of acrylic acid and methacrylic acid, as are commercially available under the names Merquat®100 (poly(dimethyldiallylammonium chloride)) and Merquat®550 (dimethyldiallylammonium chloride-acrylamide copolymer);

vinylpyrrolidone-vinylimidazolium methochloride copolymers, as are offered for sale under the names Luviquat® FC 370, FC 550, FC 905 and HM 552;

the polymers known by the name Polyquaternium-24 (commercial product for example Quatrisoft® LM 200); and

the polymers known by the names Polyquaternium-2, Polyquaternium-17, Polyquaternium-18 and Polyquaternium-27 with quaternary nitrogen atoms in the polymer main chain.

More preferred cosmetic agents are characterized in that they contain at least one polymeric quaternary ammonium compound selected from the group of homo- or copolymers of methacryloyloxyethyltrimethylammonium chloride.

One particularly suitable homopolymer is poly(methacryloyloxyethyltrimethylammonium chloride), which may if desired be crosslinked, with the INCI name Polyquaternium-37. Such products are commercially available for example under the names Rheocare® CTH (Cosmetic Rheologies) and Synthalen® CR (3V Sigma). Crosslinking may, if desired, proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

This homopolymer is preferably used in the form of a nonaqueous polymer dispersion which should have a polymer fraction of no less than 30 wt. %. Such polymer dispersions are commercially available under the names Salcare® SC 95 (approx. 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Preferred copolymers of methacryloyloxyethyltrimethylammonium chloride contain acrylamide, methacrylamide, acrylic acid C₁₋₄ alkyl esters and methacrylic acid C₁₋₄ alkyl esters as nonionogenic monomer units. Acrylamide is more preferred among these nonionogenic monomers. These copolymers, as described above for the homopolymers, may also be crosslinked. A preferred copolymer is crosslinked acrylamide-methacryloyloxyethyltrimethylammonium chloride copolymer. Such copolymers, in which the monomers are present in a weight ratio of approx. 20:80, are commercially available as approx. 50% nonaqueous polymer dispersions under the name Salcare® SC 92.

Further preferred ingredients of the hair treatment agent are protein hydrolysates and/or the derivatives thereof (P). Protein hydrolysates of both plant and animal origin or marine or synthetic origin may be used.

Animal protein hydrolysates are for example elastin, collagen, keratin, silk and milk protein hydrolysates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda). Further protein hydrolysates which are preferred are of maritime origin. These include for example collagen hydrolysates from fish or algae and protein hydrolysates from mussels or pearl hydrolysates. Examples of pearl extracts suitable herein are the commercial products Pearl Protein Extract BG® or Crodarom® Pearl. Protein hydrolysates and their derivatives likewise include cationized protein hydrolysates, wherein the underlying protein hydrolysate may originate from animals, for example from collagen, milk or keratin, from plants, for example from wheat, maize, rice, potatoes, soy or almonds, from marine life forms, for example from fish collagen or algae, or biotechnologically obtained protein hydrolysates. The protein hydrolysates (P) are contained in the compositions in concentrations of about 0.001 wt. % to about 20 wt. %, preferably of about 0.05 wt. % to about 15 wt. % and particularly preferably in quantities of about 0.05 wt. % to about 5 wt. %.

The following tables show the composition of some preferred cosmetic agents (details in wt. % relative to the total weight of the cosmetic agent unless otherwise stated).

| | | | | Formula | Formula |
|---|---|---|---|---|---|
| | Formula 1 | Formula 2 | Formula 3 | 4 | 5 |
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium | | | | | |
| compound | | | | | |
| Water, misc. | ad 100 | ad 100 | ad 100 | ad 100 | ad 100 |

| | | | | Formula | Formula |
|---|---|---|---|---|---|
| | Formula 6 | Formula 7 | Formula 8 | 9 | 10 |
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium | | | | | |
| compound | | | | | |
| Water, misc. | ad 100 | ad 100 | ad 100 | ad 100 | ad 100 |

| | Formula 11 | Formula 12 | Formula 13 | Formula 14 | Formula 15 |
|---|---|---|---|---|---|
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water, misc. | ad 100 | ad 100 | ad 100 | ad 100 | ad 100 |

| | Formula 16 | Formula 17 | Formula 18 | Formula 19 | Formula 20 |
|---|---|---|---|---|---|
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water, misc. | ad 100 | ad 100 | ad 100 | ad 100 | ad 100 |

| | Formula 21 | Formula 22 | Formula 23 | Formula 24 | Formula 25 |
|---|---|---|---|---|---|
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethylammonium | 0.05 to 3.0 | 0.1 to 2.0 | 0.2 to 1.0 | 0.5 | 0.3 |
| chloride | | | | | |
| Water, misc. | ad 100 | ad 100 | ad 100 | ad 100 | ad 100 |

| | Formula 26 | Formula 27 | Formula 28 | Formula 29 | Formula 30 |
|---|---|---|---|---|---|
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethylammonium | 0.05 to 3.0 | 0.1 to 2.0 | 0.2 to 1.0 | 0.5 | 0.3 |
| chloride | | | | | |
| Water, misc. | ad 100 | ad 100 | ad 100 | ad 100 | ad 100 |

| | Formula 31 | Formula 32 | Formula 33 | Formula 34 | Formula 35 |
|---|---|---|---|---|---|
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethylammonium | 0.05 to 3.0 | 0.1 to 2.0 | 0.2 to 1.0 | 0.5 | 0.3 |
| chloride | | | | | |
| Water, misc. | ad 100 | ad 100 | ad 100 | ad 100 | ad 100 |

| | Formula 36 | Formula 37 | Formula 38 | Formula 39 | Formula 40 |
|---|---|---|---|---|---|
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethylammonium | 0.05 to 3.0 | 0.1 to 2.0 | 0.2 to 1.0 | 0.5 | 0.3 |
| chloride | | | | | |
| Water, misc. | ad 100 | ad 100 | ad 100 | ad 100 | ad 100 |

| | Formula 41 | Formula 42 | Formula 43 | Formula 44 | Formula 45 |
|---|---|---|---|---|---|
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water, misc. | ad 100 | ad 100 | ad 100 | ad 100 | ad 100 |

| | Formula 46 | Formula 47 | Formula 48 | Formula 49 | Formula 50 |
|---|---|---|---|---|---|
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water, misc. | ad 100 | ad 100 | ad 100 | ad 100 | ad 100 |

| | Formula 51 | Formula 52 | Formula 53 | Formula 54 | Formula 55 |
|---|---|---|---|---|---|
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water, misc. | ad 100 | ad 100 | ad 100 | ad 100 | ad 100 |

| | Formula 56 | Formula 57 | Formula 58 | Formula 59 | Formula 60 |
|---|---|---|---|---|---|
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water, misc. | ad 100 | ad 100 | ad 100 | ad 100 | ad 100 |

| | Formula 61 | Formula 62 | Formula 63 | Formula 64 | Formula 65 |
|---|---|---|---|---|---|
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water | 60 to 99 | 75 to 98 | 85 to 95 | 95 | 93 |
| Misc. | ad 100 | ad 100 | ad 100 | ad 100 | ad 100 |

| | Formula 66 | Formula 67 | Formula 68 | Formula 69 | Formula 70 |
|---|---|---|---|---|---|
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water | 60 to 99 | 75 to 98 | 85 to 95 | 95 | 93 |
| Misc. | ad 100 | ad 100 | ad 100 | ad 100 | ad 100 |

| | Formula 71 | Formula 72 | Formula 73 | Formula 74 | Formula 75 |
|---|---|---|---|---|---|
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water | 60 to 99 | 75 to 98 | 85 to 95 | 95 | 93 |
| Misc. | ad 100 | ad 100 | ad 100 | ad 100 | ad 100 |

| | Formula 76 | Formula 77 | Formula 78 | Formula 79 | Formula 80 |
|---|---|---|---|---|---|
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Water | 60 to 99 | 75 to 98 | 85 to 95 | 95 | 93 |
| Misc. | ad 100 | ad 100 | ad 100 | ad 100 | ad 100 |

| | Formula 81 | Formula 82 | Formula 83 | Formula 84 | Formula 85 |
|---|---|---|---|---|---|
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water | 60 to 99 | 75 to 98 | 85 to 95 | 95 | 93 |
| Misc. | ad 100 | ad 100 | ad 100 | ad 100 | ad 100 |

| | Formula 86 | Formula 87 | Formula 88 | Formula 89 | Formula 90 |
|---|---|---|---|---|---|
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water | 60 to 99 | 75 to 98 | 85 to 95 | 95 | 93 |
| Misc. | ad 100 | ad 100 | ad 100 | ad 100 | ad 100 |

| | Formula 91 | Formula 92 | Formula 93 | Formula 94 | Formula 95 |
|---|---|---|---|---|---|
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water | 60 to 99 | 75 to 98 | 85 to 95 | 95 | 93 |
| Misc. | ad 100 | ad 100 | ad 100 | ad 100 | ad 100 |

| | | | | | Formula |
|---|---|---|---|---|---|
| | Formula 96 | Formula 97 | Formula 98 | Formula 99 | 100 |
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Quaternary | 0.05 to 3.0 | 0.1 to 2.0 | 0.2 to 1.0 | 0.5 | 0.3 |
| ammonium compound | | | | | |
| Protein hydrolysate | 0.01 to 3.0 | 0.02 to 1.5 | 0.05 to 1.0 | 0.08 | 0.8 |
| Water | 60 to 99 | 75 to 98 | 85 to 95 | 95 | 93 |
| Misc. | ad 100 | ad 100 | ad 100 | ad 100 | ad 100 |

| | Formula | Formula | Formula | Formula | Formula |
|---|---|---|---|---|---|
| | 101 | 102 | 103 | 104 | 105 |
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water | 60 to 99 | 75 to 98 | 85 to 95 | 95 | 93 |
| Misc. | ad 100 | ad 100 | ad 100 | ad 100 | ad 100 |

| | Formula | Formula | Formula | Formula | Formula |
|---|---|---|---|---|---|
| | 106 | 107 | 108 | 109 | 110 |
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water | 60 to 99 | 75 to 98 | 85 to 95 | 95 | 93 |
| Misc. | ad 100 | ad 100 | ad 100 | ad 100 | ad 100 |

| | Formula | Formula | Formula | Formula | Formula |
|---|---|---|---|---|---|
| | 111 | 112 | 113 | 114 | 115 |
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water | 60 to 99 | 75 to 98 | 85 to 95 | 95 | 93 |
| Misc. | ad 100 | ad 100 | ad 100 | ad 100 | ad 100 |

| | Formula | Formula | Formula | Formula | Formula |
|---|---|---|---|---|---|
| | 116 | 117 | 118 | 119 | 120 |
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water | 60 to 99 | 75 to 98 | 85 to 95 | 95 | 93 |
| Misc. | ad 100 | ad 100 | ad 100 | ad 100 | ad 100 |

| | | | | | For- |
|---|---|---|---|---|---|
| | Formula | Formula | Formula | Formula | mula |
| | 121 | 122 | 123 | 124 | 125 |
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Water | 60 to 99 | 75 to 98 | 85 to 95 | 95 | 93 |
| Misc. | ad 100 | ad 100 | ad 100 | ad 100 | ad |
| | | | | 100 | |

| | | | | | For- |
|---|---|---|---|---|---|
| | Formula | Formula | Formula | Formula | mula |
| | 126 | 127 | 128 | 129 | 130 |
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 131 | 132 | 133 | 134 | 135 |
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 136 | 137 | 138 | 139 | 140 |
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 141 | 142 | 143 | 144 | 145 |
| Copolymer A | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Ethanol | 0.1 to 10 | 0.5 to 8.0 | 2.0 to 6.0 | 3.2 | 1.7 |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 146 | 147 | 128 | 149 | 150 |
| Copolymer A¹⁾ | 0.05 to 10 | 0.1 to 7.0 | 0.2 to 5.0 | 0.4 | 2.7 |
| Copolymer B | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Ethanol | 0.1 to 10 | 0.5 to 8.0 | 2.0 to 6.0 | 3.2 | 1.7 |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 151 | 152 | 153 | 154 | 155 |
| Copolymer A | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Ethanol | 0.1 to 10 | 0.5 to 8.0 | 2.0 to 6.0 | 3.2 | 1.7 |
| Water | 60 to 99 | 75 to 98 | 85 to 95 | 95 | 93 |
| Misc. | ad 100 | ad 100 | ad 100 | ad 100 | ad |
| | | | | | 100 |

| | | | | | For- |
|---|---|---|---|---|---|
| | Formula | Formula | Formula | Formula | mula |
| | 156 | 157 | 158 | 159 | 160 |
| Copolymer A¹⁾ | 0.05 to 10 | 0.1 to 7.0 | 0.2 to 5.0 | 0.4 | 2.7 |
| Copolymer B²⁾ | 0.05 to 10 | 0.1 to 7.0 | 0.2 to 5.0 | 0.8 | 0.5 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Protein | 0.01 to 3.0 | 0.02 to 1.5 | 0.05 to 1.0 | 0.08 | 0.8 |
| hydrolysate | | | | | |
| Ethanol | 0.1 to 10 | 0.5 to 8.0 | 2.0 to 6.0 | 3.2 | 1.7 |
| Water | 60 to 99 | 75 to 98 | 85 to 95 | 95 | 93 |
| Misc. | ad 100 | ad 100 | ad 100 | ad 100 | ad |
| | | | | | 100 |

¹⁾Copolymer A, which consists in a proportion of at least 90 wt. % and in particular of at least 95 wt. % of structural units (A1), (A2), (A3) and (A4), wherein R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100; R2 in formula (A2) denotes a residue —(CH₂)₃—N(CH₃)₂; one residue R3 denotes —CH₂CH₃ and one residue R3 in formula (A3) denotes H.
²⁾Copolymer B, which consists in a proportion of at least 90 wt. % and in particular of at least 95 wt. % of structural unit (B1), in which the residue R4 in formula (B1) denotes —CH₃.

As explained above, the agents contemplated herein have advantageous conditioning and hair-fixing properties. The present disclosure therefore also provides a corresponding method for temporarily deforming keratinic fibers, in which a composition contemplated herein is applied onto the keratinic fibers. The present disclosure also provides use of a cosmetic agent contemplated herein for temporarily deforming keratinic fibers.

The agents contemplated herein are suitable in particular for use during heat-assisted, long-lasting hair shaping, in particular at about 80° C. to about 250° C., more preferably at about 120° C. to about 200° C. It is in turn preferable for the heat source to take the form of a heated solid, heated in particular to about 80° C. to about 250° C., more preferably to about 120° C. to about 200° C., brought into direct contact with the hair. This is the case in particular when using waving irons or hair straighteners. Uses or methods contemplated herein are preferred which are characterized in that shaping of the keratinic fibers proceeds with exposure to heat from a heat source, wherein the heat source exhibits a temperature of about 50 to about 200° C.

The cosmetic agents contemplated herein are preferably applied to the keratinic fibers prior to heat-assisted, long-lasting hair shaping.

When using curling irons, a strand of hair is wound around an appropriately heated rounded body, for example a rod-shaped or tubular body, and unwound again after a given length of time, in particular 10 to 30 seconds.

Preferred uses or methods are characterized in that the hair is shaped by straightening using hair straighteners. Mechanical straightening is understood herein to mean stretching curly hair to make the hair fibers as long as possible.

In an alternative embodiment, preferred uses or methods are characterized in that the hair is shaped into waves or curls using curling tongs or a hairdryer with diffuser.

1. A cosmetic agent containing in a cosmetically acceptable carrier
a) at least one copolymer A with at least one structural unit (A1), at least one structural unit (A2), at least one structural unit (A3) and at least one structural unit (A4), 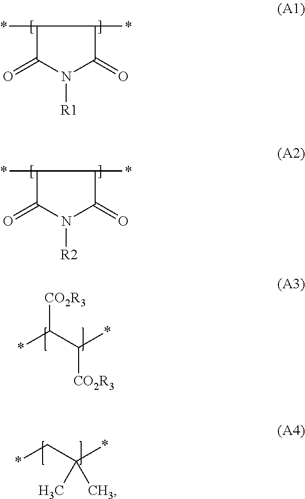
wherein
R1 denotes an optionally heterofunctionalized alkyl residue;
R2 denotes an optionally heterofunctionalized alkyl residue differing from R1;
R3 denotes an optionally heterofunctionalized alkyl residue mutually independently differing from R1 and R2
b) at least one copolymer B differing from copolymer A with at least one structural unit (B1) 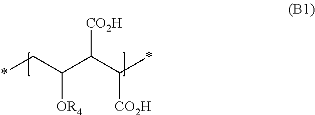
wherein R4 denotes an optionally heterofunctionalized alkyl residue; and
c) at least one quaternary ammonium compound.

2. The cosmetic agent according to claim 1, wherein the
R1 in formula (A1) denotes a residue —CH(CH₃)CH₂—(OCH(CH₃)CH₂)ₓ(O[CH₂]₂)_{y}OCH₃, in which x and y mutually independently have a value between 1 and 100;
R2 in formula (A2) denotes a residue —(CH₂)₃—N(CH₃)₂;
one residue R3 denotes —CH₂CH₃ and one residue R3 in formula (A3) denotes H.

3. The cosmetic agent according to claim 1, wherein the proportion by weight of copolymer A in the total weight of the cosmetic agent amounts to about 0.05 to about 10 wt. %.

4. The cosmetic agent according to claim 1, wherein the residue R4 in formula (B1) denotes —CH₃.

5. The cosmetic agent according to claim 1, wherein the proportion by weight of copolymer B in the total weight of the cosmetic agent amounts to about 0.05 to about 10 wt. %.

6. The cosmetic agent according to claim 1, wherein the cosmetic agent furthermore contains at least one 1,2-diol.

7. The cosmetic agent according to claim 1, wherein the quaternary ammonium compound is selected from the group of trimethylalkylammonium halides.

8. The cosmetic agent according to claim 1, wherein the proportion by weight of the quaternary ammonium compound in the total weight of the cosmetic agent amounts to from about 0.05 to about 3.0 wt. %.

9. (canceled)

10. A method for temporarily deforming keratinic fibers, the method comprising the steps of:
applying onto the keratinic fibers a cosmetic agent containing in a cosmetically acceptable carrier
a) at least one copolymer A with at least one structural unit (A1), at least one structural unit (A2), at least one structural unit (A3) and at least one structural unit (A4), 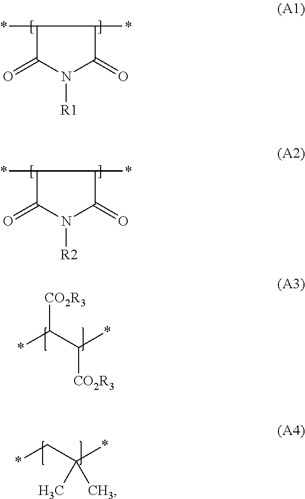
wherein
R1 denotes an optionally heterofunctionalized alkyl residue;
R2 denotes an optionally heterofunctionalized alkyl residue differing from R1;
R3 denotes an optionally heterofunctionalized alkyl residue mutually independently differing from R1 and R2
b) at least one copolymer B differing from copolymer A with at least one structural unit (B1) 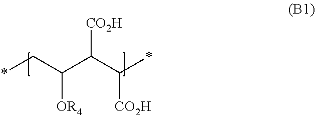
wherein R4 denotes an optionally heterofunctionalized alkyl residue; and
c) at least one quaternary ammonium compound.

11. The method of claim 10, further comprising contacting the keratinic fibers with heat.

12. The cosmetic agent according to claim 3, wherein the proportion by weight of copolymer A in the total weight of the cosmetic agent amounts to about 0.1 to about 7.0 wt. %.

13. The cosmetic agent according to claim 12, wherein the proportion by weight of copolymer A in the total weight of the cosmetic agent amounts to about 0.2 to about 5.0 wt. %.

14. The cosmetic agent according to claim 5, wherein the proportion by weight of copolymer B in the total weight of the cosmetic agent amounts to about 0.1 to about 7.0 wt. %.

15. The cosmetic agent according to claim 14, wherein the proportion by weight of copolymer B in the total weight of the cosmetic agent amounts to about 0.2 to about 5.0 wt. %.

16. The cosmetic agent according to claim 6, wherein the cosmetic agent furthermore contains 1,2-octanediol.

17. The cosmetic agent according to claim 8, wherein the proportion by weight of the quaternary ammonium compound in the total weight of the cosmetic agent amounts to from about 0.1 to about 2.0 wt. %.

18. The cosmetic agent according to claim 17, wherein the proportion by weight of the quaternary ammonium compound in the total weight of the cosmetic agent amounts to from about 0.2 to about 1.0 wt. %.
